(19) United States
(12) Plant Patent Application Publication
(10) Pub. No.: US 2005/0241028 P1
(43) Pub. Date: Oct. 27, 2005

(54) 'Haas Cherry' cultivar prunus avium Haas

(50) PRUNUS AVIUM; CHERRY

(76) Inventors: Richard Wellington, Geneva, NY (US); John Wellington, Geneva, NY (US)

Correspondence Address: International Plant Management, Inc., 55826 60th Avenue, Lawrence, MI, 49064, US

(21) Appl. No.: 10/829,690

(22) Filed: Apr. 23, 2004

(51) Int. Cl. A01H 5/00

(52) U.S. Cl. PLT/181

(57) ABSTRACT

A new and distinct variety of sweet cherry tree, ‘Haas Cherry’, originating as the result of a cross between ‘Giant’, an unpatented selection, and ‘Emperor Francis’, an unpatented selection. This new variety is unique because its tree has proven to be reliable to produce regular, heavy crops of mid-season, dark red, fleshed fruit that are dually suitable for commercial uses in both processing markets as well as direct retail sales.

### FIELD OF THE INVENTION

A new and distinct cultivar of sweet cherry tree originated as a seedling of Prunus avium in the breeding program of Cornell University and is hereinafter referred to as ‘Haas’ sweet cherry. This new variety is unique because its tree has proven to be reliable to produce regular, heavy crops of mid-season, dark red, fleshed fruit that are dually suitable for commercial uses in both processing markets as well as direct retail sales of fresh fruits to on-farm customers who pick their own fruit in Eastern states of North America. It possesses another unique feature in that it will pollinate both ‘Emperor Francis’ and ‘Ulster’, the two most important mid-season processing sweet cherry varieties in the Eastern United States sweet cherry processing industry. Here to for, no effective pollinator variety has been available for this purpose, which could be relied upon to also yield heavily itself.

### SUMMARY OF THE INVENTION

This new and distinct variety of sweet cherry was discovered by Dr. Richard Wellington (deceased), a plant breeder at the New York State Agricultural Experiment Station, Geneva, N.Y., a research unit of Cornell University, hereinafter referred to as Geneva Experiment Station. By breeding methodology convention at the Geneva Experiment Station it was designated NY 1725. This selection was initially assigned Experiment Station Breeding Record Number 40118, a hybrid population of trees that resulted from hybridizing the varieties ‘Giant’×‘Emperor Francis’ in 1940 and this seedling was designated as NY 1725 when it was planted in 1944 as part of a population of 16 siblings possessing the same parentage. The orchard location where the seedling was grown and first noticed was designated as Orchard Number 27, Row 1, Tree 34. This seedling was selected because in 1951 Cornell scientists purposely looked for and chose seedlings with heavy cropping characteristics for the relatively unfavorable weather conditions existing for sweet cherry production at Geneva, N.Y., where both cold winters and spring frosts frequently cause serious damage to trees and flower buds of many varieties of sweet cherries. This seedling also was chosen due to its tolerance to rain cracking of its fruits compared to the ‘Bing’ variety, which has predominated in western USA sweet cherry marketing for well over half a century. NY 1725 was propagated on Mazzard seedling rootstocks in 1952 so as to create more trees for further evaluations on the Geneva Experiment Station. Subsequently it has been propagated on Mahaleb seedling and ‘Gisela 6’ rootstocks and remains true to the description herein.

### A BRIEF DESCRIPTION OF THE DRAWINGS

The accompanying photographs show typical specimens of the new variety as depicted in color as nearly true as is reasonably possible in color illustrations of this character. These specimens were obtained at the Geneva Experiment Station, Geneva, N.Y.

FIG. 1. Illustrates two intact fruit at maturity with size dimensions and fruit shape depicted accurately.

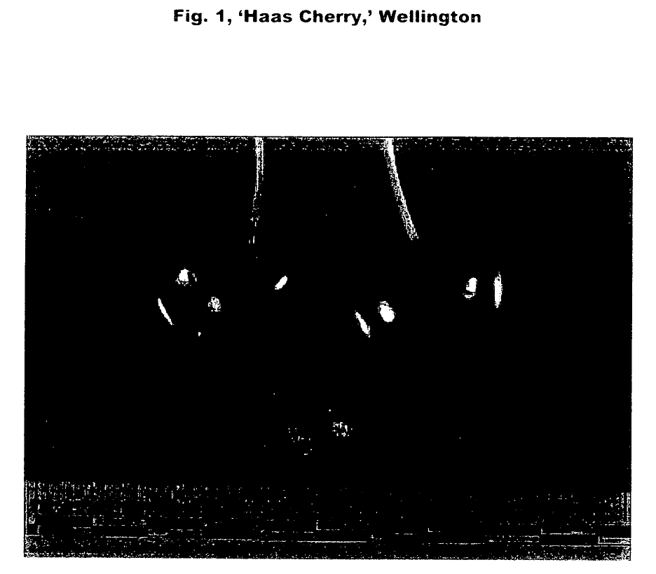

### A BOTANICAL DESCRIPTION OF THE PLANT

A detailed description of the ‘Haas cherry’ cultivar follows using The Royal Society of London Colour identification except where general color terms are sufficient.
Parentage: A hybrid seedling of the cross: ‘Giant’×‘Emperor Francis’. Locality of the original discovery and observations is Orchard Number 27, Row 1, Tree 034 (assigned the designation NY 1725), Geneva Experiment Station, Geneva, N.Y., U.S.A.
Tree:
   Age and rootstock of specimen.—16 years old on Mazzard seedling.
   Height.—5.0 M.
   Width.—4.5 M.
   Trunk diameter.—21.5 cm at 100 cm above ground level.
   Vigor.—Medium.
   Density.—Medium.
   Form.—Spreading, laterals are moderately upright to about the same degree as ‘Emperor Francis’, the male parent of the new variety.
   Production.—Heavy, over 200 pounds per season per tree on Mazzard seedling rootstocks.
   Bearing.—Annual on spurs and on 2 to 4 basal buds of one year old shoots.
   Disease resistance/tolerance.—Similar to ‘Emperor Francis’ in tolerance to brown rot fungus of the blossoms and fruit (rated 7 at Geneva on scale of 1, as worst, to 9, as best); also similar to ‘Emperor Francis’ in tolerance to bacterial canker of the tree and fruit (rated as 7 for tree and 8 for fruit, again with 1, as worst, and 9, as best; highly tolerant of black knot fungus of the wood.
   Cold hardiness.—Excellent, crops regularly in test orchards in commercial fruit districts of New York, Pennsylvania, and Michigan where mid-winter temperatures often go below −15 degrees Fahrenheit. Tree livability is excellent in commercial sweet cherry orchard districts of the Eastern U.S.A. and is rated by Geneva Experiment Station pomologists as similar to that of its male parent, “Emperor Francis”.
   Frost tolerance.—Very good (rated as 7 at Geneva on scale of 1, as worst, to 9 as best)
   Graft compatibility.—Very good, produces smooth compatible graft unions with Mazzard seedling, Mahaleb seedling, and slight overgrowth (as is usually the case for all sweet cherry scion varieties) with ‘Gisela 6’ cherry rootstocks.
Trunk:
   Size.—21.5 cm in diameter at 100 cm above the ground for 16 year old tree on Mazzard seedling rootstock.
   Surface.—Bark is smooth with prominent lenticels.
   Bark color.—Grayed Purple 183 B, where directly exposed to the sun.
   Lenticels.—Grayed Orange 167 B, varying in length from 0.75 to 3 cm and width from 0.25 to 0.6 cm, prominent, horizontal, elliptical.
Vegetative buds:
   Placement.—At bud positions 4 and higher number from the base of new growth, and at the tip of each fruit spur.
   Appearance.—Pointed and flatter than flower buds on previous season's annual growth of shoots.
   Color.—Grayed Orange 165B in fully dormant stage of development.
Leaves:
   Size.—9.0 to 11.5 cm in length, 5.5 to 6.5 cm in width.
   Form.—Symmetrical halves on both sides of central axis.
   Thickness.—Medium.
   Texture.—Crisp, leathery.
   Pubescence.—None on either surface.
   Margin.—Serrations regular and bluntly pointed.
   Adaxial surface.—Green 137A.
   Abaxial surface.—Yellow Green 148C.
   Veination.—Pinnate.
   Vein color.—Adaxial Yellow Green 144D, abaxial Yellow Green 144B.
   Petiole.—6.0 to 6.5 cm length.
   Petiole color.—Red Purple 59A.
   Petiole groove.—Narrow.
   Glands.—1 to 2 on petiole, bluntly globose, length 1.5 to 1.75 mm, width 1 to 1.25 mm, color Orange 28A.
Flowers:
   Blooming period.—Mid-season, April 25 to May 1 in Geneva, New York, similar to ‘Emperor Francis’ an unpatented selection.
   Presentation.—Non-showy, white.
   Fragrance.—Sweet, flowery.
   Fertility.—Self-incompatible, belonging to the pollination compatibility group designated as having S₁S₄ alleles, which are the same as those possessed by ‘Rainier’, ‘Hudson’ and ‘Sylvia’, hence, making them incompatible with each other.
   Pollen.—Present, plentiful, good pollen source for pollenizing all other sweet cherries except those with identical S alleles, as stated above.
   Corolla diameter.—32 mm.
   Number of flowers per cluster.—5 to 8.
   Petals.—Single, round, length 10 mm, width 10 mm, 5 in number, slightly overlapping, margin slightly ruffled, soft texture, White 155D.
   Peduncle.—Length 4.8 to 5.2 mm, width 0.4mm, Green 143B.
   Filament.—Length 8 mm, width 0.1 mm, White 155 D.
   Anther.—Profuse pollen, Yellow 13A
   Sepals.—5 in number, Green 143 B, bluntly ovate in shape.
Fruit:
   Maturity when described.—commercial ripeness, 19 degrees brix.
   Date of first picking.—Mid-season ripening, July 4 to July 10 at Geneva, New York.
   Size.—Weight 8.5 g, length 3.0 cm, width 2.7 cm.
   Form.—Cordate appearance.
   Stem cavity.—Wide and tolerant to concentric rain-induced cracking.
   Stem.—length 38 mm, width 1.25 mm, color Green 143 B.
Skin:
   Thickness.—Medium.
   Tendency to crack.—Low to moderate, similar to ‘Emperor Francis, an unpatented selection, rated as 7 at Geneva (1, worst, to 9 best).
   Color.—Grayed Purple 187A.
   Flavor.—Very Good.
Flesh:
   Texture.—Somewhat above average in firmness, rated as 6.5 at Geneva (1, worst, to 9 best).
   Acidity.—Medium until sugar levels get to above mid-teen levels in degrees brix.
   Flavor.—Good.
   Aroma.—Present, pleasing.
   Color.—Grayed Purple 187B.
   Coloration in the pit cavity.—Same as flesh.
   Eating quality.—Very good when mature to above mid-teen levels of degrees brix and excellent when eaten at high-teen levels of degrees brix.
Stone:
   Size.—2 cm in length, 1.75 cm in width at the widest point near the center, 1.1 cm in width at the widest point of the flatter direction.
   Sides.—Ridges, 3 in number, with varying amounts of encircling of the suture side of the pit.
   Type.—Semi-freestone a commercial maturity.
   Tendency to crack.—Non-existent in nature.
Use:
   Dual purpose.—fresh marketing for on-farm marketing of pick-your-own fruit is well suited by this variety because it bears heavy, annual crops of good flavored fruit with minimal rain-induced cracks; and processing for bakery and dairy uses of frozen, pitted fruit is also well suited because of heavy yielding characteristics of this variety under relatively adverse climatic conditions found in processing sweet cherry districts of the Eastern U.S.A. This variety will be highly sought after by processing cherry growers in the Eastern U.S.A. to be used as a highly effective pollinator for commercial, processing orchards of ‘Emperor Francis’, an unpatented selection, and ‘Ulster’, an unpatented selection, the two most widely planted, processing sweet cherry varieties grown in the Eastern U.S.A.

1. ‘Haas’, a new and distinct variety of sweet cherry tree, Prunus avium, substantially as herein shown and described, characterized as to novelty by the unique combination of hardy, highly productive tree with successful pollination compatibility with all varieties except those having the S₁S₄ alleles.
